(19) United States
(12) Patent Application Publication
(10) Pub. No.: US 2017/0348553 A1
(43) Pub. Date: Dec. 7, 2017

(54) EMULSION COMPOSITION CONTAINING A COLD CREAM

(71) Applicant: Henkel AG & Co. KGaA, Duesseldorf (DE)

(72) Inventors: Heike Schelges, Willich (DE); Barbara Heide, Krefeld (DE); Clemens Riecke, Remscheid (DE); Martina Kampmann, Korschenbroich (DE)

(73) Assignee: Henkel AG & Co. KGaA, Duesseldorf (DE)

(21) Appl. No.: 15/616,137

(22) Filed: Jun. 7, 2017

(30) Foreign Application Priority Data
Jun. 7, 2016 (DE) 10 2016 209 979.5

(51) Int. Cl. A61Q 19/00; A61K 8/34; A61K 8/06; A61K 8/73; A61K 8/97; A61K 8/92

(52) U.S. Cl. CPC A61Q 19/005; A61K 8/97; A61K 8/925; A61K 8/062; A61K 8/73; A61K 8/34; A61K 8/922

(57) ABSTRACT

A cosmetic emulsion composition for skincare, which comprises
a. at least one anionic surfactant and/or a fatty acid soap,
b. at least one animal wax and/or a derivative of an animal waxes,
c. at least one vegetable oil,
d. at least one C₂-C₆ polyol
e. at least one natural thickener
f. at least one extract from chamomile and
g. allantoin,
   has excellent skin care properties (skin-soothing effect, moisturizing properties). Moreover, they are storage stable and have a pleasant texture.

### CROSS-REFERENCE TO RELATED APPLICATION

This application claims priority to German Application No. 10 2016 209 979.5, filed Jun. 7, 2016, which is hereby incorporated in its entirety by reference.

### TECHNICAL FIELD

The present disclosure relates to a cosmetic emulsion composition for skin care based on a specific active ingredient mixture comprising animal waxes, vegetable oils, polyols, natural thickeners, chamomile extract and allantoin, to the use of this specific active ingredient mixture for improving the care properties, stability and/or texture of cosmetic emulsion compositions, as well as to a method for skin care, for which a cosmetic composition is applied to the skin, in particular to the facial skin.

### BACKGROUND

The human skin, as the largest organ of man, performs numerous vital functions. With an average surface area of about 2 m² in the adult human body, it plays a prominent role as a protective and sensory organ. The purpose of this organ is to mediate and counteract mechanical, thermal, actinic, chemical and biological stimuli. It also plays a significant role as a regulatory and target organ in human metabolism. The cosmetic treatment of the skin therefore is an important component of human body care. The purpose of skin care in the cosmetic sense is to strengthen or restore the natural function of the skin as a barrier against weather and environmental influences and against the loss of body-specific materials (such as water, natural fats, electrolytes) and to slow down the natural aging process in order to impart a young and healthy appearance to the skin.

Of particular interest therefore are active cosmetic ingredients for the treatment of the skin, which provide caring properties which protect against or revitalize aging phenomena. Preparations for skin care and skin protection, which contain a whole series of different active cosmetic ingredients or active ingredient complexes, are offered in large numbers and in many forms of preparation.

The so-called cold creams, which have been produced for many centuries and are essentially based on a mixture of vegetable or animal waxes, oils and water, have long been known. However, the products were extremely unstable and quickly became rancid.

In the course of time, ways were found to stabilize cold creams, the use of a mixture of mineral oil and borax being most effective in cold creams. However, all the products had the disadvantage that they had an undesirably heavy consistency and left a highly unaesthetic fatty film on the treated skin.

In the application EP 929290A2, the use of borax was dispensed with and, instead, a combination of water, polyols, polyisobutene and silicones was processed into a cold cream. In this way, it was possible to develop lighter, clear products, which are outstandingly suitable for cleaning in the sense of removing make-up.

However, the incorporation of (even modern) cold creams in emulsion bases in a satisfactory manner was not always possible. In addition, there is an increasing demand for natural, mild, nourishing cleansing products, in which the use of borax, mineral oils and/or synthetic oils is to be dispensed with.

The object of the present disclosure therefore is to provide a basic cosmetic formulation comprising the components of classical cold creams. The formulation is to have an excellent texture and very good stability and storage properties. Furthermore, the compositions should also provide moisture to the skin and ideally have a soothing effect, especially on sensitive and/or irritated skin.

### BRIEF SUMMARY

Cosmetic emulsion compositions for skin care and methods employing the same are provided herein. In an embodiment, a cosmetic emulsion composition for skin care includes:

a) at least one animal wax and/or a derivative of an animal wax,

b) at least one vegetable oil,

c) at least one C₂-C₆ polyol,

d) at least one natural thickener,

e) at least one extract from chamomile, and

f) allantoin.

In another embodiment, a method includes applying a cosmetic emulsion composition to skin. The emulsion composition includes:

a) at least one animal wax and/or a derivative of an animal wax,

b) at least one vegetable oil,

c) at least one C₂-C₆ polyol

d) at least one natural thickener

e) at least one extract from chamomile and

f) allantoin.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it has now been found that compositions comprising at least one natural thickener in addition to a combination of four specific active care ingredients (animal waxes, vegetable oils, chamomile flower extract and allantoin) in a polyol- and a water-containing base, accomplish the abovementioned tasks. Cosmetic emulsion compositions based on this combination are distinguished from conventional compositions of the prior art by a good cosmetic skin compatibility, applicability and texture. In addition, the emulsion compositions are also physically and chemically stable over a long period of time. Furthermore, the emulsion compositions have very good care properties: after application, they leave behind a non-greasy, smooth skin sensation and, in particular, maintain the moisture balance of the skin. Moreover, it was possible to sooth irritated, reddened and/or itchy skin by the application of the emulsion compositions.

The stability of the rheological properties of the cosmetic composition and its stability against phase separation, including stability against the sedimentation of suspended solids, are referred to as the physical stability. The term “chemical stability” refers to the stability against chemical transformation or decomposition, as can be tested, for example, by the activity test of the cosmetically active substances d).

A first object of this application is a cosmetic emulsion composition including:

a) at least one animal wax and/or a derivative of an animal waxes,
b) at least one vegetable oil,
c) at least one C₂-C₆ polyol,
d) at least one natural thickener,
e) at least one extract from chamomile, and
f) allantoin.

The cosmetic emulsion compositions may be present in the form of a liquid or flowable oil-in-water emulsion, water-in-oil emulsion or multiple emulsion, especially in the form of an oil-in-water-in-oil emulsion or a water-in-oil-in-water emulsion, macro emulsion, miniemulsion, micro emulsion, PIT emulsion, nano emulsion, Pickering emulsion and/or a hydrodispersion. Preferably they are present in the form of a liquid or flowable oil-in-water emulsion.

In the event that the cosmetic emulsion compositions are present as an oil-in-water emulsion, they comprise an aqueous portion. Preferably, the emulsion composition is a viscous liquid. Preferred cosmetic emulsion compositions are characterized by a water content of from about 30 to about 90% by weight, preferably of from about 35 to about 85% by weight, particularly of from about 40 to about 80% by weight and especially of from about 45 to about 75% by weight, based on the total weight of the emulsion.

Suitable components a) for the purposes of the present disclosure may preferably be selected from beeswax (Cera alba) or other insect waxes, spermaceti, wool wax (lanolin), rump fat and/or the derivatives of these waxes. Preferably wax derivatives modified with polyalkylene glycol and/or polyglycerol come into consideration as suitable wax derivatives since, in addition to their care properties, they may support the emulsification of vegetable oils b) in the emulsion compositions. The at least one animal wax and/or wax derivative a) should preferably be a solid at 25° C. in order to achieve optimal stability of the emulsion compositions and have a melting range from about 45° C.-85° C., more preferably from about 50° C.-80° C. and in particularly from about 55° C. to about 75° C. Particularly preferred for the purposes of the present disclosure is beeswax modified with polyalkylene glycol and/or polyglycerol, wherein the beeswax derivatives, known under the INCI designations PEG-(2-25) beeswax and/or polyglyceryl-(2-10) beeswax are particularly preferred.

Particularly preferably, the emulsion compositions contain beeswax or one of the beeswax derivatives known under the INCI names of PEG-3 beeswax, PEG-8 beeswax and/or polyglyceryl-3 beeswax, preferably beeswax (Cera alba) as component a). The proportion by weight of the at least one animal wax and/or wax derivative a) of the emulsion compositions preferably is from about 0.01 to about 10.0% by weight, particularly from about 0.05 to about 5.0% by weight and especially from about 0.10 to about 3.0% by weight, based on the total weight of the emulsion compositions. In a first preferred embodiment, emulsion compositions are preferred which contain beeswax (Cera alba) or another insect wax, spermaceti, wool wax (lanolin), rump fat and/or a derivative of these waxes as animal wax and/or wax derivative. Within this embodiment, emulsion compositions are particularly preferred, which contain, as animal wax and/or wax derivative a), beeswax and/or at least one of the beeswax derivatives, which are known under the INCI names of PEG-(2-25) Beeswax and/or polyglyceryl (2-10) Beeswax. In this embodiment, emulsion compositions are particularly preferred, which contain as animal wax and/or wax derivative a) beeswax (Cera alba) or one of the beeswax derivatives, which are known under the INCI names of PEG-3 Beeswax, PEG-8 Beeswax and/or Polyglyceryl-3 Beeswax; preferably, they contain beeswax.

For the purpose of the present disclosure, preferably the following oils come into consideration as vegetable oils b) Coconut oil, (sweet) almond oil, walnut oil, peach kernel oil, apricot kernel oil, avocado oil, tea tree oil, soybean oil, cottonseed oil, sesame oil, sunflower oil, tsubaki oil, chamomile oil, evening primrose oil, rice bran oil, palm oil, palm kernel oil, mango kernel oil, cranberry oil, sea buckthorn oil, meadowfoam seed oil, safflower oil, macadamia nut oil, grape seed oil, amaranth seed oil, argan oil, bamboo oil, olive oil, wheat germ oil, pumpkin seed oil, mallow oil, hazelnut oil, castor oil, safflower oil, corn oil, olive oil, rapeseed oil, canola oil, sasanqua oil, jojoba oil, rambutan oil, cocoa butter and/or shea butter. Especially preferred are soybean oil, cottonseed oil, sunflower oil, palm oil, palm kernel oil, linseed oil, safflower oil, wheat germ oil, peach kernel oil, cranberry oil, sea buckthorn oil and/or coconut oil, sunflower oil being especially preferred.

It was possible to achieve optimum care results and excellent stability of the emulsion compositions as contemplated herein when, as a care agent mixture, a combination of

beeswax (Cera alba), PEG-3 beeswax, PEG-8 beeswax and/or polyglyceryl-3 beeswax wax and

soybean oil, cottonseed oil, sunflower oil, palm oil, palm kernel oil, linseed oil, almond oil, castor oil, corn oil, olive oil, rapeseed oil, sesame oil, soybean oil, safflower oil, wheat germ oil, peach kernel oil, cranberry oil, sea buckthorn oil and/or coconut oil; especially

beeswax (Cera alba) and sunflower seed oil was used.

The percentage of the at least one vegetable oil b) preferably is from about 0.01 to about 20.0% by weight, particularly from about 0.05 to about 15.0% by weight and especially from about 0.10 to about 10.0% by weight of the total weight of the emulsion compositions.

In a second preferred embodiment, emulsifying compositions are preferred, which contain soybean oil, cottonseed oil, sunflower oil, palm oil, palm kernel oil, linseed oil, almond oil, castor oil, corn oil, olive oil, rapeseed oil, sesame oil, soybean oil, safflower oil, wheat germ oil, peach kernel oil, cranberry oil, sallow thorn oil and/or coconut oil and, in particular, sunflower oil, as vegetable oil b).

In the context of the present disclosure, suitable C₂-C₆-polyols c) preferably are polyols, which contain 2 to 6 carbon atoms, preferably 2 to 4 carbon atoms, and 2 to 6 hydroxyl groups, preferably 2 to 3 hydroxyl groups in their molecule.

Examples of preferred C₂-C₆ polyols d) are: ethylene glycol, 1,2-propylene glycol 1,3-propylene glycol, dipropylene glycol, 2-methyl-1,3-propanediol, 1,2-butylene glycol, 1,3-butylene glycol, 1,4-butylene glycol, isoprene glycol, 1,2-pentanediol, 1,3-pentanediol, 1,2-hexanediol, 1,6-hexanediol, 1,2,6-hexanetriol, glycerol, diglycerol, erythritol, sorbitol or mixtures of these polyols. Water-soluble alcohols are especially preferred. Especially preferred are glycol, 1,2-propylene glycol, 1,3-propylene glycol, 2-methyl-1,3-propanediol, glycerol, 1,2-butylene glycol, 1,3-butylene glycol, 1,4-butylene glycol, pentylene glycol, 1,2-hexanediol, 1,6-hexanediol, 1,2,6-hexanetriol, dipropylene glycol, diglycerol, erythritol, sorbitol as well as mixtures of the aforementioned substances. Very particular preference is given to 1,2-propylene glycol, glycerol, 1,3-butylene glycol, 1,6-hexanediol, dipropylene glycol and/or diglycerol and, in particular, mixtures of glycerol and 1,6-hexanediol. The percentage of the at least one C₂-C₆ polyols c) preferably is from about 1.0 to about 25.0% by weight, particularly from about 2.0 to about 20.0% by weight and especially from about 3.0 to about 15.0% by weight of the total weight of the emulsion compositions.

In a third preferred embodiment, preference is given to emulsion compositions, which contain, as C₂-C₆ polyol c), 1,2-propylene glycol, glycerol, 1,3-butylene glycol, 1,6-hexanediol, diglycerol and/or dipropylene glycol, mixtures of glycerin and 1,6-hexanediol being preferred.

Natural thickeners d) for the purposes of the present disclosure preferably are natural hydrocolloids. It has been found that natural hydrocolloids are particularly well suited for stabilizing the care products a) and b) in a base of water and polyols c) over a long period of time. In addition, the use of natural hydrocolloids in the emulsion compositions leads to emulsions with a very pleasant texture.

Examples of preferred natural thickeners d) are:

polysaccharides, such as glucanan; modified and unmodified starches; amylose; amylopectin; dextrans; cellulose and the derivatives thereof (methylcellulose, hydroxyalkylcellulose, ethylhydroxyethylcellulose and carboxymethylcellulose); mannans; xylans; lignins; chitin; chitosans; pectins; alginic acids and alginates; arabinogalactans; carageenans and carrageenates; agar; gums (gum arabic, karaya); locust bean gum; galactomannans such as guar gum and non-ionic derivatives thereof (hydroxypropyl guar); xanthan gum; scleroglucan; and mixtures thereof.

It was possible to achieve particularly good results with regard to stability and texture of the mixtures of emulsion compositions when at least one carrageenan and/or carrageenate was used as the natural thickener d).

The percentage of the at least one natural thickener e) preferably is from about 0.01 to about 3.00% by weight, particularly from about 0.10 to about 2.00% by weight and especially from about 0.20 to about 1.50% by weight of the total weight of the emulsion compositions.

In a fourth preferred embodiment, emulsion compositions are preferred, which contain the following as at least one natural thickener d): polysaccharides such as glucanan; modified and unmodified starches; amyloses; amylopectin; dextrans; cellulose and the derivatives thereof (methylcellulose, hydroxyalkylcellulose, ethylhydroxyethylcellulose and carboxymethylcellulose); mannans; xylans; lignins; chitin; chitosans; pectins; alginic acids and alginates; arabinogalactans; carageenans and carrageenates; agar; gums (arabicum, karaya); carob bean gum; galactomannans such as guar gum and the nonionic derivatives thereof (hydroxypropyl guar); xanthan gum; scleroglucan; and mixtures thereof. Particularly preferred emulsion compositions within this embodiment contain carrageenans and/or carrageenates as natural thickeners.

As a further active ingredient, the emulsion compositions contain at least an extract of chamomile e), which preferably is used in a percentage of from about 0.001 to about 1.0% by weight, particularly preferably of from about 0.003 to about 0.75% by weight and in particular of from about 0.005 to about 0.5% by weight of the total weight of the emulsion compositions.

The extract of chamomile may be obtained by known manufacturing methods, for example by grinding all parts or individual parts of the chamomile (such as leaves, flowers, roots), and, if appropriate, subsequently drying and/or extracting. The extraction usually takes place at elevated temperatures in an extractor (such as a Soxhlet). Suitable solvents are used for the extraction, depending on whether the oil-soluble or water-soluble components of the plant are required.

For example, water or alcohol as well as the mixtures thereof can be used as extracting agent for producing said plant extracts. As alcohols, for example, lower alcohols such as ethanol and isopropanol, especially however also multi-hydric alcohols such as ethylene glycol, propylene glycol, butylene glycol, glycerin and/or sorbitol come into consideration and moreover as sole extracting agent or also in a mixture with water. For example, plant extracts based on water/alcohol(s) in a ratio of from about 1:10 to about 10:1 have proven to be particularly suitable.

Due to their excellent skin-relaxing effect, extracts of chamomile flowers are particularly suitable for the cleaning agent as contemplated herein. These may be oil-based, aqueous or aqueous alcoholic. Corresponding extracts are commercially available in the mixture with the respective extraction agent from various suppliers, for example, from companies such as Cosmetochem, Symrise or Croda.

In a fifth preferred embodiment, preference is given to emulsion compositions, which comprise, as an extract from chamomile e), an aqueous, aqueous-alcoholic or oil-based chamomile flower extract in an amount of from about 0.001 to about 1.0% by weight, preferably of from about 0.003 to about 0.75% % and, in particular of from about 0.005 to about 0.5% by weight of the total weight of the emulsion compositions.

In a sixth preferred embodiment, preference is given to emulsion compositions, which comprise an extract from allantoin f) in an amount of from about 0.001 to about 1.0% by weight, preferably of from about 0.003 to about 0.75% % and, in particular of from about 0.005 to about 0.5% by weight of the total weight of the emulsion compositions.

It was found that the combination of the active ingredients a), b), e) and f) form a highly effective skin care complex in the emulsion compositions, which regenerates not only dry, sensitive and/or irritated skin, but gives it more moisture and smoothness and also relieves stresses and/or itching.

Particular preference is given to a care complex which (based on the total weight of the cosmetic emulsion compositions) includes

From about 0.10 to about 3.0% by weight of beeswax (Cera alba),

From about 0.10 to about 10.0% by weight of sunflower oil,

From about 0.005 to about 0.5% by weight chamomile flower extract and

From about 0.01 to about 0.5% by weight of allantoin.

The stability and texture of, in particular, liquid or flowable emulsion compositions as contemplated herein may be improved further by the addition of one or more stabilizing agents.

Higher fatty alcohols h) and/or thickeners g), especially homopolyacrylic acid polymers, have been found to be particularly suitable additional stabilizing agents.

In the context as contemplated herein, higher fatty alcohols h) are linear or branched, saturated or unsaturated alcohols having 16 to 30, preferably 18 to 26 and particularly 20 to 24 carbon atoms.

Particularly preferred are arachidyl alcohol, behenyl alcohol and/or lignoceryl alcohol, and particular preference is given to behenyl alcohol.

The higher fatty alcohol(s) (h) may preferably be present in the emulsion compositions in an amount of from about 0.1 to about 20.0% by weight, particularly preferably of from about 0.5 to about 15.0% by weight and, in particular, of from about 1.0 to about 10.0% by weight of the total weight of the emulsion compositions.

In a further preferred embodiment, the emulsion compositions as contemplated herein furthermore comprise a higher alcohol, preferably behenyl alcohol, in an amount of from about 0.1 to about 20.0% by weight, particularly preferably of from about 0.5 to about 15.0% by weight and in particular of from about 1.0 to about 10.0% by weight of the total weight of the emulsion compositions.

Suitable additional thickeners g) preferably are homopolyacrylic acid or salts thereof. These may be cross-linked or not cross-linked. Cross-linked polyacrylic acid polymers are understood to be those, to which a certain percentage of bi-functional or multifunctional monomers are added during the polymerization. Preferred crosslinking agents are ethylene glycol dimethacrylate, 1,9-decadiene, divinylbenzene and allyl ethers of pentaerythritol, sucrose, ethylene glycol and propylene glycol. Suitable polymeric compounds are, for example, commercially available under the trademark Carbopol® and are known under the INCI name of Carbomer.

The additional thickener(s) (g), particularly homopolyacrylic acids or salts, may preferably be present in the emulsion compositions in an amount of from about 0.05 to about 3.0% by weight, particularly preferably of from about 0.10 to about 2.0% by weight and, in particular, of from about 0.20 to about 1.5% by weight of the total weight of the emulsion compositions.

In a further preferred embodiment, the emulsion compositions as contemplated herein furthermore comprise an additional thickener g), especially a homopolyacrylic acid (salt) in an amount of from about 0.05 to about 3.0% by weight, particularly preferably of from about 0.10 to about 2.0% by weight and in particular of from about 0.20 to about 1.5% by weight of the total weight of the emulsion compositions.

Aside from the aforementioned essential and optional components, the cosmetic emulsion compositions may contain at least one further active care ingredient, preferably a skin conditioning active ingredient from the group of further oil components i) and/or the vitamins j).

The oil phase preferably is present in the cosmetic emulsion compositions in an amount of from about 0.50 to about 50.00% by weight of the total weight of the emulsion compositions, more preferably of from about 1.00 to about 40.00% by weight, particularly preferably of from about 1.50 to about 30.00% by weight and most preferably of from about 2.00 to about 25.00% by weight. The emulsifier (s) is (are) not counted either as belonging to the oil phase or to the water phase.

Preferred additional oil components i), which can be used in emulsion compositions as contemplated herein, are, for example, selected from:

volatile silicone oils, which may be cyclic, such as, for example, octamethylcyclotetrasiloxane, decamethylcyclopentasiloxane and dodecamethylcyclohexasiloxane, and mixtures thereof, such as are contained, for example, in the commercial products DC 244, 245, 344 and 345 from Dow Corning, or linear, such as hexamethyldisiloxane (L 2), octamethyltrisiloxane (L 3), decamethyltetrasiloxane (L 4), mixtures of any two or three of L 2, L 3 and/or L 4, such as are contained, for example, in the commercial products DC 2-1184, Dow Corning® 200 (0.65 cSt), and Dow Corning® 200 (1.5 cSt) of Dow Corning,

non-volatile higher molecular weight linear dimethylpolysiloxanes, commercially obtainable, for example, under the name Dow Corning® 190, Dow Corning® 200 fluids with viscosities ranging from about 5-100 cSt, preferably from about 5-50 cSt or even from about 5-10 cSt, and Baysilon® 350 cM,

the esters of linear or branched saturated or unsaturated fatty alcohols having 2 to 30 carbon atoms with linear or branched saturated or unsaturated fatty acids having 2 to 30 carbon atoms, which may be hydroxylated. These include 2-ethylhexyl palmitate (e.g. Cegesoft® C 24), hexadecyl stearate (Eutanol® G 16), hexyldecyl laurate, isodecyl neopentanoate, isononyl isononanoate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-butyl stearate, n-hexyl laurate, n-decyl oleate, oleyl oleate, oleyl erucate, erucyl oleate, erucyl erucate, ethylene glycol dioleate and ethylene glycol dipalmitate,

The benzoates of linear or branched C₈-22 alkanols, for example the commercial products Finsolv® TN (C_{12-C15} alkyl benzoate), Finsolv® SB (isostearyl benzoate) and Finsolv® EB (ethylhexyl benzoate),

the C₈-C₂₂ fatty alcohol esters of monohydric or polyhydric C₂-C₇ hydroxycarboxylic acids, in particular the esters of glycolic acid, lactic acid, malic acid, tartaric acid, citric acid and salicylic acid. Such esters based on linear C_{12/15} alkanols, such as C₁₂-C₁₅ alkyl lactate, and C_{12/13} alkanols branched in the 2-position, for example di-C₁₂-C₁₃ alkyl malate, are obtainable under the trademark Cosmacol® from Nordmann, Rassmann GmbH & Co, Hamburg, in particular the commercial products Cosmacol® EMI, Cosmacol® ESI and Cosmacol® ETI,

the addition products of ethylene oxide and/or propylene oxide to monohydric or polyhydric C₃-20 alkanols such as butanol, butanediol, myristyl alcohol and stearyl alcohol, such as PPG-14 butyl ether (Ucon Fluid®, PPG-9 butyl ether (Breox® B25), PPG 10 butanediol (Macol® 57), PPG 3 myristyl ether (Witconol® APM) and PPG-15 stearyl ether (Arlamol® E),

liquid paraffin oils, isoparaffin oils, such as the commercial products of the Permethyl® series, in particular isododecane, isohexadecane and isoeicosan,

synthetic hydrocarbons, such as polyisobutene or polydecene and alicyclic hydrocarbons, for example, the commercial product 1,3-di-(2-ethylhexyl) cyclohexane (Cetiol® S),

the symmetrical, asymmetric or cyclic esters of carbonic acid with fatty alcohols, for example glycerol carbonate, dicapryl carbonate (Cetiol® CC),

dicarboxylate esters of linear or branched C₂-C₁₀ alkanols, in particular diisopropyl adipate, di-n-butyl adipate, di-(2-ethylhexyl) adipate, dioctyl adipate, diethyl/di-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate,

di-n-alkyl ethers having a total of 12 to 36 and, in particular 12 to 24 carbon atoms, such as di-n-octyl ether (Cetiol® OE), di-n-hexyl n-octyl ether and n-octyl-n-decyl ether.

Particularly preferred oils are silicone oils and esters of linear or branched saturated or unsaturated fatty alcohols with 2 to 30 carbon atoms with linear or branched saturated or unsaturated fatty acids having from 2 to 30 carbon atoms, in particular, volatile and non-volatile silicone oils from the Dow Corning 200 Fluid series and/or Dow Corning 9041, the ester oils 2-ethylhexyl palmitate, hexyldecyl laurate, 2-ethylhexyl stearate, isopropyl myristate, isopropyl palmitate and 2-ethylhexyl laurate and/or the benzoates of linear or branched C₈-22 alkanols, in particular the commercial product Finsolv®.

For some embodiments of the emulsion compositions as contemplated herein, it may also be preferred to use mixtures of the aforementioned oils. Mixtures of two types of oil components, such as a silicone oil and an ester oil, are particularly preferred.

Preferably the following vitamins, provitamins and vitamin precursors as well as their derivatives are understood to be suitable “vitamins”:

Vitamin A: the group of substances, referred to as vitamin A, include retinol (vitamin A1) and 3,4-didehydroretinol (vitamin A2). β-Carotene is the provitamin of the retinol. Suitable vitamin A components, which come into consideration, are, for example, vitamin A acid and the esters thereof, vitamin A aldehyde and vitamin A alcohol, and the esters thereof, such as the palmitate and the acetate.

Vitamin B: among others, the following belong to the vitamin B group or to the vitamin B complex

vitamin B₁ (thiamine)

vitamin B₂ (riboflavin)

vitamin B₃. The compounds, nicotinic acid and nicotinic acid amide (niacinamide) are frequently listed under this name.

vitamin B₅ (pantothenic acid and panthenol). In this group panthenol, is preferably used.

Derivatives of panthenol, which can be used are, in particular:
esters and ethers of panthenol, pantolactone as well as cationically derivatized panthenols. Individual representatives are, for example, panthenol triacetate, panthenol monoethyl ether and the monoacetate thereof as well as cationic panthenol derivatives.

Vitamin B₆ (pyridoxine as well as pyridoxamine and pyridoxal).

Vitamin C (ascorbic acid): use in the form of the palmitate ester, the glucoside or the phosphate may be preferred. The use in combination with tocopherol may also be preferred.

Vitamin E (tocopherols, especially α-tocopherol).

Vitamin F: The term “vitamin F” is usually understood to mean essential fatty acids, particularly linoleic acid, linolenic acid and arachidonic acid.

Vitamin H: The compound (3aS,4S, 6aR)-2-oxohexa-hydrothienol[3,4-d]imidazole-4-valeric acid is designated as vitamin H, but in the meantime the trivial name biotin has gained acceptance.

Vitamins, provitamins and vitamin precursors from the groups A, B, E and H are especially preferred. Nicotinic acid amide, biotin, pantolactone and/or panthenol are particularly preferred; nicotinic acid amide is more particularly preferred. The amount of vitamin(s), vitamin derivative(s) and/or the vitamin precursor(s) preferably is from about 0.005 to about 2.00% by weight, more preferably from about 0.006 to about 1.50% %, particularly preferably from about 0.008 to about 1.00% by weight and in particular from about 0.01 to about 0.50% by weight of the total weight of the cosmetic emulsion compositions.

Further active ingredients, auxiliaries and additives, which may be used in the cosmetic emulsions as contemplated herein, are, for example:

UV filters,

structurants such as maleic acid and lactic acid,

dyes for coloring the material,

substances for adjusting the pH, such as α- and β-hydroxycarboxylic acids such as citric acid, lactic acid, malic acid and glycolic acid,

complexing agents such as EDTA, NTA, β-alanine diacetic acid and phosphonic acids,

Ceramides. Ceramides are understood to be N-acylsphingosine (fatty acid amides of sphingosine) or synthetic analogues of such lipids (so-called pseudo-ceramides),

propellants such as propane-butane mixtures, N₂O, dimethyl ether, carbon dioxide and air,

antioxidants,

perfume components,

preservatives,

A second object as contemplated herein is the cosmetic use of a combination comprising

a) at least one animal wax and/or a derivative of an animal wax,
b) at least one vegetable oil,
c) at least one C₂-C₆ polyol
d) at least one natural thickener
e) at least one extract from chamomile and
f) allantoin.
in skin care agents, preferably in cosmetic emulsions for

improvement of care;

improvement of the skin-soothing effect;

improvement of the moisturizing properties;

improvement of the stability; and

improvement of the texture of the skin care products.

A further object as contemplated herein is a method for cosmetic skin care for which a cosmetic emulsion composition is applied on the skin.

For the preferred embodiments of the contemplated uses and of the contemplated method, what has been said in connection with the contemplated materials applies mutatis mutandis.

### Examples

The following cosmetic emulsion compositions were prepared (the quantities are given in % by weight)

| | Body | Body | Body | Body |
|---|---|---|---|---|
| | Lotion I | Lotion II | Butter I | Butter II |
| Sunflower oil | 5.00 | | 1.00 | |
| Olive oil | | 5.00 | | 1.00 |
| 1,6-Hexanediol | 5.00 | 5.00 | 6.00 | 5.00 |
| Glycerin | 3.00 | 3.00 | 5.00 | 6.00 |
| Beeswax (Cera alba) | 0.15 | | 0.15 | |
| PEG-8 Beeswax | | 0.20 | | 0.10 |
| Carrageenan | 0.20 | | 0.10 | |
| Xanthan Gum | | 0.10 | | 0.30 |
| Chamomilla recutita | 0.007 | 0.01 | 0.10 | 0.05 |
| matricaria Flower extract | | | | |
| Allantoin | 0.01 | 0.05 | 0.05 | 0.01 |
| Ethylhexyl palmitate | 5.00 | 5.00 | | |
| Sodium stearoyl lactylate | 0.20 | 0.20 | 0.20 | 0.20 |
| Behenyl alcohol | 2.00 | 2.00 | 10.00 | 9.00 |
| Shea butter | 1.00 | 1.00 | | 1.00 |
| Carbomer | 0.30 | 0.30 | 0.50 | 0.40 |
| Tocopherol | 0.03 | 0.03 | 0.03 | 0.03 |
| Preservatives | q.s. | q.s. | q.s. | q.s. |
| pH adjusting agent (pH | q.s. | q.s. | q.s. | q.s. |
| 4.7 to 5.5) | | | | |
| Water | ad 100 | ad 100 | ad 100 | ad 100 |

While at least one exemplary embodiment has been presented in the foregoing detailed description, it should be appreciated that a vast number of variations exists. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic emulsion composition for skin care, comprising
a) at least one animal wax and/or a derivative of an animal wax,
b) at least one vegetable oil,
c) at least one C₂-C₆ polyol,
d) at least one natural thickener,
e) at least one extract from chamomile, and
f) allantoin.

2. The cosmetic emulsion composition of claim 1, wherein the at least one animal wax and/or the derivative thereof a) is selected from beeswax (Cera alba) or other insect waxes; spermaceti; wool wax (lanolin); rump fat; and/or the derivatives of these waxes.

3. The cosmetic emulsion composition according to claim 1, wherein
the at least one animal wax and/or the derivative thereof a) is selected from beeswax; and/or from at least one of the beeswax derivatives, known under the INCI name of PEG-(2-25) beeswax; and/or of polyglyceryl-(2-10) beeswax or one of the beeswax derivatives known under the INCI name of PEG-3 Beeswax; of PEG-8 beeswax; and/or Polyglyceryl-3 beeswax.

4. The cosmetic emulsion composition according to claim 1, wherein
the at least one animal wax and/or the derivative a) thereof is present in amounts of from about 0.01 to about 10% by weight, based on the total weight of the emulsion composition.

5. The cosmetic emulsion composition according to claim 1, wherein
the at least one vegetable oil is selected from soybean oil, cottonseed oil, sunflower oil, palm oil, palm kernel oil, linseed oil, almond oil, castor oil, corn oil, olive oil, rapeseed oil, sesame oil, soybean oil, safflower oil, wheat germ oil, peach kernel oil, cranberry oil, sea buckthorn oil and/or coconut oil.

6. The cosmetic emulsion composition according to claim 1, wherein
the at least one vegetable oil b) is present in amounts of from about 0.01 to about 20.0% by weight, based on the total weight of the emulsion composition.

7. The cosmetic emulsion composition according to claim 1, wherein
the at least one C₂-C₆ polyol c) is selected from the group of glycol, 1,2-propylene glycol, 1,3-propylene glycol, 2-methyl-1,3-propanediol, glycerol, 1,2-butylene glycol, 1,3-butylene glycol, 1,4-butylene glycol, pentylene glycol, 1,2-hexanediol, 1,6-hexanediol, 1,2,6-hexanetriol, dipropylene glycol, diglycerol, erythritol, sorbitol and mixtures of the aforementioned substances.

8. The cosmetic emulsion composition according to claim 1, wherein
the at least one C₂-C₆ polyol c) is present in amounts of from about 0.01 to about 25.0% by weight, based on the total weight of the emulsion composition.

9. The cosmetic emulsion composition according claim 1, wherein
the at least one natural thickener d) is selected from polysaccharides, modified starches, unmodified starches, amylose, amylopectin, dextrans, celluloses, derivatives of cellulose, mannans, xylans, lignins, chitin, chitosans, pectins, alginic acids and alginates, arabinogalactans, carageenans, carrageenates, agar, gums (gum arabic, karaya), locust bean gum, galactomannans, xanthan gums, scleroglucan, and mixtures thereof.

10. The cosmetic emulsion composition according claim 1, wherein
the at least one natural thickener d) is present in amounts of from about 0.01 to about 3.0% by weight, based on the total weight of the emulsion composition.

11. The cosmetic emulsion composition according to claim 1,
comprising as extract of chamomile f), an aqueous, aqueous-alcoholic or oil-based chamomile flower extract which, based on the total weight of the emulsion composition, is present in amounts of from about 0.001 to about 1.0% by weight, based on the total weight of the emulsion composition.

12. The cosmetic emulsion composition according to claim 1,
comprising the allantoin f) in amounts of from about 0.001 to about 1.0% by weight, based on the total weight of the emulsion composition.

13. The cosmetic emulsion composition according to claim 1, wherein
the composition comprises at least one additional thickener.

14. The cosmetic emulsion composition according to claim 1, wherein
the composition furthermore comprises at least one linear fatty alcohol h.

15. A method comprising applying a cosmetic emulsion composition to skin, wherein the emulsion composition comprises
a) at least one animal wax and/or a derivative of an animal wax,
b) at least one vegetable oil,
c) at least one C₂-C₆ polyol
d) at least one natural thickener
e) at least one extract from chamomile and
f) allantoin.

16. The cosmetic emulsion composition according to claim 1, wherein
the at least one animal wax and/or the derivative a) thereof is present in amounts of from about 0.10 to about 3% by weight, based on the total weight of the emulsion composition;
the at least one vegetable oil b) is present in amounts of from about 0.10 to about 10.0% by weight, based on the total weight of the emulsion composition;
the at least one natural thickener d) is present in amounts of from about 0.20 to about 1.50% by weight, based on the total weight of the emulsion composition;
the extract of chamomile f) comprises an aqueous, aqueous-alcoholic or oil-based chamomile flower extract which, based on the total weight of the emulsion composition, is present in amounts of from about 0.005 to about 0.5% by weight, based on the total weight of the emulsion composition.

17. The cosmetic emulsion composition according to claim 1, wherein
the at least one C₂-C₆ polyol c) is selected from the group of 1,2-propylene glycol, glycerol, 1,3-butylene glycol, 1,6-hexanediol, diglycerol, dipropylene glycol, or mixtures of the aforementioned substances; and
wherein the at least one C₂-C₆ polyol c) is present in amounts of from about 3.0 to about 15.0% by weight, based on the total weight of the emulsion composition.

18. The cosmetic emulsion composition according to claim 13, wherein the at least one additional thickener g) is present in amounts of from about 0.05 to about 3.0% by weight, based on the total weight of the emulsion composition.

19. The cosmetic emulsion composition according to claim 13, wherein the composition comprises a homopolyacrylic acid or the salts thereof as thickener g) present in amounts of from about 0.20 to about 1.50% by weight, based on the total weight of the emulsion composition.

20. The cosmetic emulsion composition according to claim 14, wherein the composition comprises a linear fatty alcohol having 18 to 26 carbon atoms as the at least one linear fatty alcohol h) present in amounts of from about 1.0 to about 10.0% by weight, based on the total weight of the emulsion composition.
